(19) United States
(12) Patent Application Publication
(10) Pub. No.: US 2018/0168498 A1
(43) Pub. Date: Jun. 21, 2018

(54) Computer Automated Method and System for Measurement of User Energy, Attitude, and Interpersonal Skills

(71) Applicant: Analytic Measures Inc., Palo Alto, CA (US)

(72) Inventors: Jared Christopher Bernstein, Palo Alto, CA (US); Jian Cheng, Palo Alto, CA (US)

(21) Appl. No.: 15/380,913

(22) Filed: Dec. 15, 2016

(51) Int. Cl. A61B 5/16; G10L 25/63; G06F 3/0488; G06F 3/16; G06F 3/01; G09B 5/00; A61B 5/11; A61B 3/113

(52) U.S. Cl. CPC A61B 5/165; G10L 25/63; G06F 3/0488; G06F 3/167; G06F 3/017; A61B 2562/0219; G09B 5/00; A61B 5/1107; A61B 3/113; G06F 2203/011; A61B 5/167

(57) ABSTRACT

A person's suitability for many activities is manifest and made evident more in a sample of the person's spontaneous verbal and other voluntary behavior than in any traditional written document such as a resume or a certificate of educational or vocational qualification. For many social and commercial roles, a spontaneous positive outlook, an appropriate level of energy, and coherent, considerate spoken communication are key behavior elements that interviewers look for. Embodiments disclosed include improved systems and methods of extracting sentiment and estimating affect from speech-borne features, by capturing and incorporating other, non-speech, voluntary actions in response to a set of performance tasks and combining these non-speech parameter values with the content and manner of verbal behavior to produce more accurate estimates of expected human reaction to the performance samples within a given performance period and to derive accurate estimates from smaller intervals of a person's performance.

### CROSS-REFERENCE TO RELATED APPLICATIONS

NA

### BACKGROUND AND RELATED ART

Computer automated systems and methods that improve the accuracy of automatic estimates of affect and reduce the time needed to make such estimates are disclosed.

Traditional Method: Effective interviews reveal a sample of candidate's psychological traits and/or attribute profile in ways that are not explicit in a resume or other prepared text. Skilled interviewers look for good attitude, high energy and social communication skills that predict success—especially in project groups and in customer-facing jobs. These characteristics emerge in a skilled interview, but the interview process is relatively expensive and often inconsistent. Interviewing remains a vast, inefficient cottage industry.

Current computer automated methods and systems of interviewing only partially address the inconsistency. Most current affect analysis works from text, speech, and/or facial images, and has mainly been applied in evaluation of human reaction to specific external events, candidates, products, or actions. That is, the evaluation is often focused on predicting the reaction of a population of voters or consumers to something on offer.

In another tradition, developers of psychological tests have developed tasks and questions with corresponding scoring rubrics that are designed to measure a person's traits, which are enduring characteristics or propensities to certain kinds of actions and reactions across a variety of circumstances. Psychological tests are validated, in part, by the consistency of the score produced when the test is given to the same person on two or more occasions. Developers remove task items that do not elicit consistent response patterns within persons across time.

A person's spontaneous speech offers a unique window into that person's most natural modes of thought (see e.g. Klesse et al., 2015). Consumer research on preference, modality, and self-control indicates that people reliably express more self-indulgent and emotionally driven choices in speaking than in writing or in any form of manual selection. Speech is the modality of interviews—skilled interviewers don't just listen to what you say, but they notice how you say it and how quickly it comes out.

In considering characteristics of candidate responses during job interviews, Murphy (2012) found that levels of “attitude” and “energy” are most predictive of job tenure and success, regardless of occupation. Attitude and energy are working labels for traits that may share as much as half their variance in the working population. Attitude varies positive-negative, and energy varies high-low. Attitude certainly shares variance with more traditional psychological constructs such as valence, emotion, sentiment, and affect. Energy is similar to what psychologists might call arousal or psychomotor activation or motivation. Detecting and estimating levels of “attitude” and “energy” is often inconsistent at best, and is extremely inaccurate at worst.

Attitude and energy are reflected in facial expression, in the content and dynamics of speech, and in bodily posture and motion (trunk, head, hands, eyes). Laboratory and commercial technologies exist that measure emotion and arousal with some degree of accuracy from facial images, sometimes combined with voice, motion, or text materials (Swinton & El Kaliouby, 2012, Metallinou et al., 2015).

Relevant theory, methods, and results come from several disciplines with many interwoven sub-fields: machine learning, sentiment analysis, spoken language processing, effective computing, Human Computer Interfacing (HCI), psychometrics, bio-behavioral analytics, multimodal interaction, emotion detection, consumer research, psychological testing, and computational linguistics.

In the speech literature, emotion is sometimes conceived as belonging to one of a set of categories: e.g. anger, disgust, fear, joy, sadness or surprise. Schuller et al. (2011) and Moataz et al. (2011) reviewed the work on recognition of emotions from speech signals. Commonly used acoustic cues for speech-borne emotion include global and temporally local prosodic features, such as the means, standard deviations, ranges, and maximum, minimum or median values of pitch, energy, and fundamental frequency (F0). This is described in papers by Murray & Arnott (1993), Dellaert et al. (1996), Rosenfeld et al. (2003), Yildirim et al. (2004), Mower et al. (2011), and Asgari et al. (2014). Further, Ivanov & Chen (2012) articulate that analysis of modulation spectra of such spectral features is effective in identifying personality traits of speakers. Some of this research has also tracked inter-word silence, vowel/consonant articulation, and similar phonetic information, which requires automatic segmentation, based on automatic speech recognition (ASR) alignment. For example, Yildirim et al. (2004) used such speech features successfully as evidence of four intended emotions (sadness, anger, happiness, and neutral) as expressed in speech by an actress. There are some intuitive generalizations that come from these experiments: for example, anger and happiness is often accompanied in speech by longer utterance duration, shorter inter-word silence, and higher pitch and energy values with wider ranges. Methods that focus on the lexico-semantic content of speech (phrase spotters) are being applied currently to monitor call center agents, and several of the papers cited above report improved emotion-detection accuracy from combining ASR-extracted lexical information with acoustic and phonetic features [U.S. Pat. No. 7,487,094 & US Publication 20140337072 A1].

Analyzing speech for affect estimation is similar to analyzing speech for language proficiency estimation in that the accuracy of the estimate is not very sensitive to differences in ASR accuracy as measured by word error rate. Reasonably accurate affect estimates from speech signals alone have been observed by Rosenfeld et al. (2004) and more recently confirmed in results reported by Ezzat et al. (2012) and Kaushik et al. (2013).

### SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a computer automated system, including a processing unit, a non-transitory storage element coupled to the processing unit and having instructions encoded thereon, which instructions when implemented by the processing unit cause the computer automated system to: capture a user input including at least one of a speech input and a touch input; combine the captured speech and touch input in at least one of linear and non-linear form, and based on the combined speech and touch input, generate a state estimate; where the generated state estimate includes extracting a single or plurality of user attributes; and based on the extracted single or plurality of user attributes, assess the user's apparent interpersonal proficiency, elements of which include expression of positive attitude, evident energy level, and/or level and sensitivity of communication skills. Other embodiments of this aspect include corresponding computer systems, apparatus, and computer programs recorded on one or more computer storage devices, each configured to perform the actions of the methods.

One general aspect includes a computer implemented method including: capturing and recording user behavior as input including at least one of a speech input and a touch input; combining feature values derived from the captured speech and touch input in at least one of linear and non-linear form, and based on the combined feature values derived from the captured speech and touch input, generating a state estimate; wherein the generated state estimate includes extracting a single or plurality of user attributes; and based on the extracted single or plurality of user attributes, assessing the user psychological state from composite attribute values.

A further computer implemented method includes capturing and recording user behavior as input including a speech input and a touch input, along with other facial and bodily movement input. The spatiotemporal features of the movement input are combined with speech and touch features in at least one of linear and non-linear form to generate an estimate of a single or plurality of user attributes. Based on the extracted single or plurality of user attributes, the computer implemented method generates an estimate of single or plurality of composite attribute values. These attributes and composite attributes are combined in estimating user characteristics including, among others, user psychological state. User behavior is registered as movement input is derived by one or more cameras and/or by other sensor-transducers that may be worn on the body or a bodily appendage. For example, a camera captures the position and movements of the head, eyes, and mouth of the user. Other embodiments of this aspect include corresponding computer systems, apparatus, and computer programs recorded on one or more computer storage devices, each configured to perform the actions of the methods.

### BRIEF DESCRIPTION OF THE DRAWINGS

The drawings illustrate the design and utility of embodiments of the present invention, in which similar elements are referred to by common reference numerals. In order to better appreciate the advantages and objects of the embodiments of the present invention, reference should be made to the accompanying drawings that illustrate these embodiments. However, the drawings depict only some embodiments of the invention, and should not be taken as limiting its scope. With this caveat, embodiments of the invention will be described and explained with additional specificity and detail through the use of the accompanying drawings in which:

FIG. 1 is a block diagram showing optimization of feature scales and combination of captured speech, touch and video according to an embodiment of the system.

FIG. 2 is a block diagram of an operational system with Attribute Calculator according to an embodiment.

FIG. 3 is a block diagram of the Attribute Calculator showing extracting or accepting feature values produced by the Spoken Language Processor (SLP), by the Touch Processor (TP), and by the Video Processor (VP) and using its matrix-vector map in generating attribute values according to an embodiment.

FIG. 4A is an overview of a flow diagram illustrating a step by step process by which Feature Calculators are determined using both a participant user's digital behavior signals and observer ratings.

FIG. 4B is an overview of a flow diagram illustrating a step by step process by which Session Interactions are automatically rated using both a participant user's digital behavior signals and an Attribute Calculator.

FIG. 5 illustrates a working embodiment of the system and method in a networked environment.

### DETAILED DESCRIPTION

In the following description, for purposes of explanation, numerous specific details are set forth in order to provide a thorough understanding of the invention. It will be apparent, however, to one skilled in the art that the invention can be practiced without these specific details.

Reference in this specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments.

Embodiments disclosed include computer automated systems and methods that combine content & manner of speaking with rate & accuracy of motor behavior to improve accuracy of automatic affect measurement. Preferred embodiments incorporate user screen-touch behavior into affect analysis and bring speech-borne affect analysis to the talent acquisition and talent management fields.

Embodiments disclosed include extended machine learning techniques that enable computer automated systems and methods to extract and assess novel sets of attributes that reveal human psychological states. Preferred embodiments include systems and methods that record speech and touch response latencies, along with rates of responding by touch or drawing (under direct instruction) in interactive tasks, and the accuracy and time-course of active touch sequences used in drawing.

Embodiments disclosed include systems and methods configured to extract and determine user attitude and energy, which extracting and determining comprises capturing speech signals, and combining the captured speech signals with motions captured on touch-screens.

Embodiments disclosed include a computer automated system, comprising a processing unit, and non-transitory storage medium or element coupled to the computer automated system and having instructions encoded thereon. The encoded instructions when implemented or executed by the processing unit cause the computer system to automatically capture touch and speech estimates via a plurality of input media. The captured touch and speech are combined in linear or/and non-linear form and based on the user state synchronous with their capture; state (attribute) estimates are generated by the computer automated system. Alternate embodiments automatically generate state estimates that progressively increase the accuracy of the said estimates based on user/respondent response time, combined in linear or/and non-linear forms. Preferably, a single or plurality of tasks, a single or plurality of response parameters, and a combination thereof, are optimized for attribute and/or state estimate accuracy. In some embodiments, the aforementioned combinations are utilized by the computer automated system to generate soft attributes like user/respondent traits and habits. According to an alternate embodiment, eye movements, like a hi-pass eye position, a temporary gaze, or a permanent gaze are also used to generate user/respondent attributes. Additional embodiments capture data from accelerometers to measure large muscle movements and combine or correlate the captured large muscle movements with the eye movements, the speech and touch responses to further enhance the accuracy of the computer automated calculations.

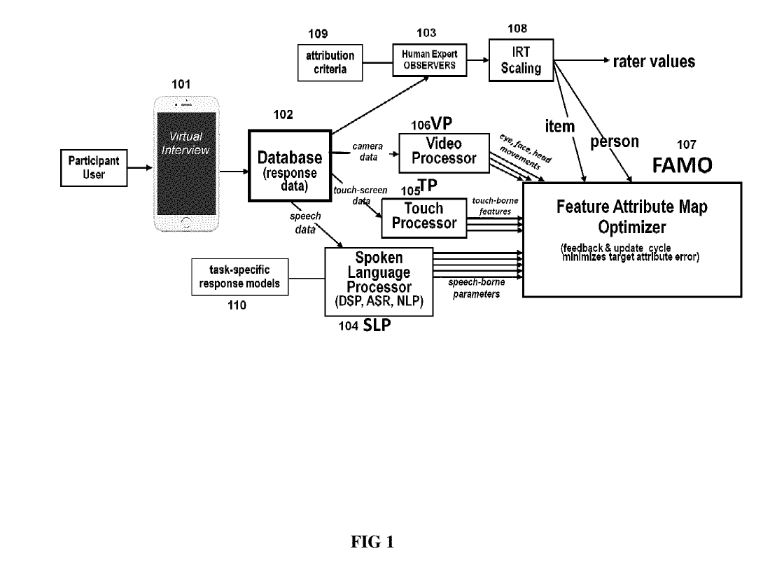

FIG. 1 is a block diagram showing optimization of feature scales and rules for combining features of captured speech, touch and video according to an embodiment of the system. Further, the figure illustrates the logic of a typical embodiment of the “training phase” set up according to an embodiment. A device or set of devices present content to a “participant user” to elicit a response or responses, i.e. a single or plurality of inputs, from the participating user. These sets of content presentations may be termed “tasks” that elicit segments of behavior. Content may be presented to the user via acoustic signals that may include spoken instructions and/or other speech that serves to define the range of appropriate responses that may be expected from users. Content may be presented in graphic form, via a graphical user interface, including but not limited to text and dynamic or animated forms on a screen, which may serve to define the range of appropriate responses that may be expected from the participant user. User responses, i.e. inputs may include speech, touch, gestures, gazes, or other voluntary movements that may comply with the explicit or implicit demands of the presentations. A single device such as a smartphone or a set of devices that may include a microphone for recording acoustic signals, a touch-sensitive screen that records user touch screen gestures, or any other device or means, capable of recording user response, already invented or yet to be invented may be deployed. Variations and modifications are possible as would be apparent to a person having ordinary skill in the art.

According to an embodiment, a set of one or more prompting events is played (preferably via the speaker and visual display) to a participant user. The analog-to-digital transducer set (microphone, touch-screen, camera, accelerometers, and other sensors) captures, and optionally records, digital signals that result from the speech and other behavior of the user. The signal capture may be continuous over the period of time that includes several or many prompting events, or the signal capture may be intermittent, occurring between prompting events. The transducer set may record the signal streams for later transmission to a database, and/or it may transmit the resulting signals directly to a database for storage. The capture, recording and transmission of the behavior-caused signals may be performed by physical and logical subsystems of a single device such as smart phone 101, enabling coordinated action in synchrony. Alternatively, they may be accomplished by separate physical and logical systems that are not co-located and may not have ongoing communication between transducers in the set.

The behavior-caused signals, captured through smart phone 101, and stored in database 102 are presented to observers or clinical experts 103 (e.g. employment recruiters or psychologists) via digital-to-analog transducers that reconstruct single or multiple aspects of user behavior in forms that enable human observers to rate segments of user behavior with reference to attributes with explicit criteria. An example of an attribute is “energetic” and criteria may include audible features of speech or visible features of a touch-screen gesture, which may give a human observer the impression that the person who produced the original speech or gesture was behaving with relatively high or low energy. Multiple judgments of one or more attributes from one or more observers may be combined 108 into a single scale to form a relatively reliable or observer-independent rating of a segment of behavior. The combining of ratings 108 may be performed in a preferred embodiment by an implementation of Item Response Theory, such as is found in the FACETS computer program (Linacre, 2003).

According to an embodiment, Spoken Language Processor (SLP) 104 accepts speech signal data from the database and produces streams of acoustic parameters and phonetic features, each as a function of time, quantized, for example, in centi-seconds. The SLP also implements a speech recognition function (see e.g. Povey et al., 2011, Zhang et al., 2014) with reference to a general or to a task-specific language model to produce a time-aligned string of words and other interspersed or superimposed linguistic and paralinguistic events such as silences, filled pauses, and voice pitch gestures (i.e. significant F0 movements). The SLP also applies a part-of-speech tagger, a parser, and a single or plurality of semantic analyzers (see e.g. Jurafsky & Martin, 2010) to produce supra-lexical units of meaning as conventional semantic units. Each linguistic unit is associated with a time that relates to its prompting event and to other co-occurring units. Touch Processor (TP) 105 accepts touch position signals from the touch screen and produces a set of features. Video processor (VP) 106 accepts camera data from database 102 and produces another set of features, each a function of time, that include gaze direction, two eye opening heights, splines that represent eyebrow contours and a spline that represents the visible positions of the user's lips and/or mouth opening. Feature Attribute Map Optimizer (FAMO) 107 uses machine learning in an update-feedback-update cycle to successively minimize the error or the weighted difference between attribute vectors produced by the human observers, or based on human observer ratings, and attribute vectors produced by the matrix-vector maps based on digital features captured by transducers and processed by SLP, TP and VP. The machine learning method can be linear regression, neural networks, support vector regressions, deep learning, or any other optimization procedure that determines a map from a matrix of feature values to a vector of attribute values.

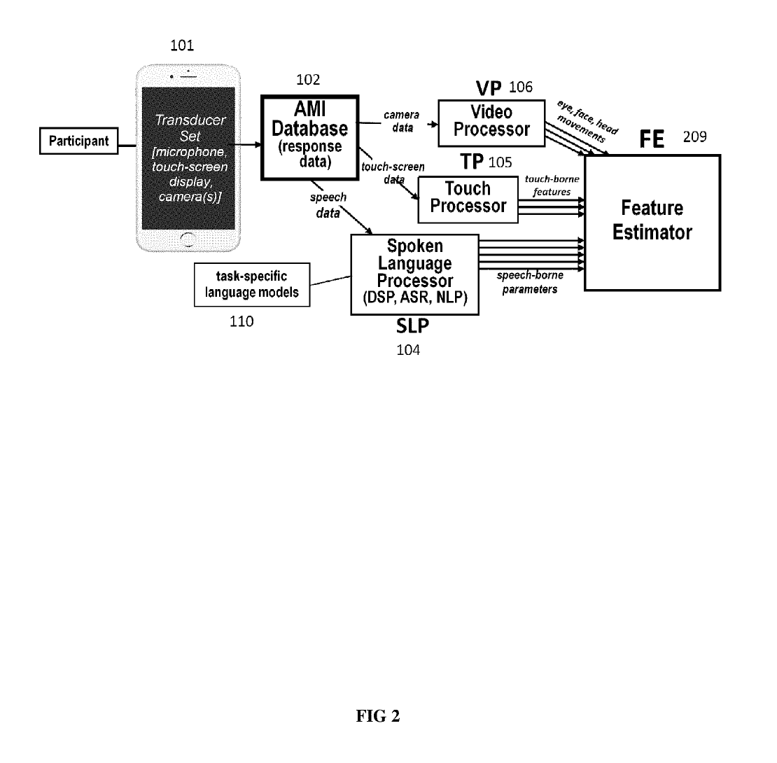

FIG. 2 is a block diagram of an operational system comprising Attribute Calculator 209 according to an embodiment. According to an embodiment, the Attribute Calculator (AC) 209 extracts time-synchronized sequences of feature vectors and nominal events from the Spoken Language Processor 104, Touch Processor (TP) 105 and Video Processor (VP) 106. The AC 209 transforms the incoming features with respect to distributions of values and delta values established by operation of the FAMO 107 during training, producing new attribute-relevant values. The new values may include means, modes, medians, quartiles, extremes, moments, segments, peaks, linear and quadratic regression coefficients, Percentiles, Durations, Onsets, and similar relevant descriptive values, as well as the spectral characteristics of these new variables as they form signals in time.

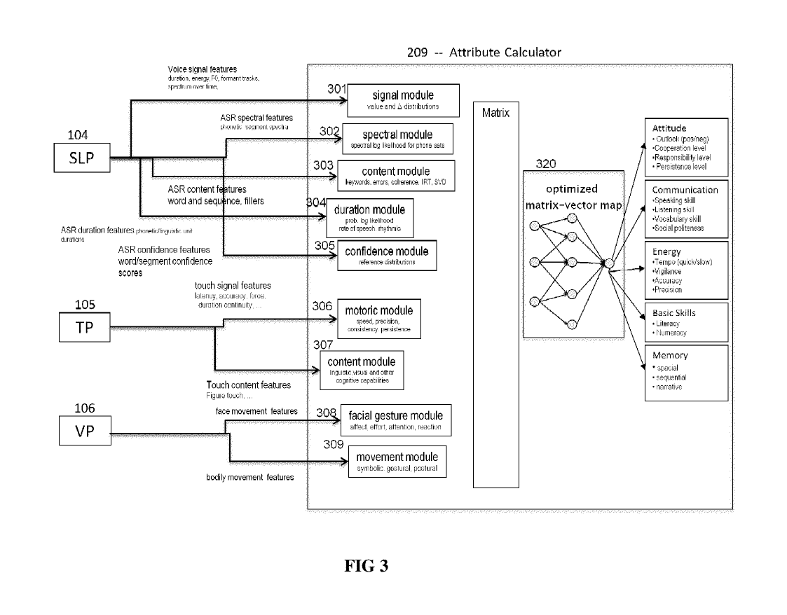

FIG. 3 is a block diagram of Attribute Calculator showing it generating feature values from combinations of the values produced by the Spoken Language Processor (SLP), by the Touch Processor (TP), and by the Video Processor (VP) according to an embodiment. The Attribute Calculator 209 implements a function developed in the Feature Attribute Map Optimizer (FAMO) 107 for mapping a sequence of feature vectors produced by the Spoken Language Processor, the Touch Processor and the Video Processor into a vector of attributes 320, which can be combined into composite attributes of a behavior sample. According to an embodiment, the Attribute Calculator (AC) 209 extracts time-synchronized sequences of feature vectors from the Spoken Language Processor 104, including voice signal features that may include waveforms, voice activity durations, energy, glottal period durations, fundamental frequency (F0), formant frequency and bandwidth tracks, frequency spectra, and similar basic, time varying parameters of an acoustic speech signal, including energy, loudness, Mel Frequency Cepstral Coefficient (MFCC), Perceptual Linear Prediction (PLP), Probability of Voicing, jitter, Shimmer, and Harmonics-to-Noise Ratio, frequency band energies, and spectral tilt. The AC 209 transforms the incoming features with respect to distributions of values and delta values established by operation of the FAMO 107 during training, producing new attribute-relevant values. The new values may include means, modes, medians, quartiles, extremes, trio segments, peaks, linear and quadratic regression coefficients, Percentiles, Durations, Onsets, and similar relevant descriptive values, as well as the spectral characteristics of these new variables as they form signals in time. The transforming operations of the signal-modeling module 301 produce new sets of parameters, which form part of the time-sequence matrix of values that form the input to the optimized matrix-vector map 320. For basic signal values such as maximum and mean values over a time segment, which do not vary over successive frames of a behavior segment, a single value may be repeated in the corresponding places in the output parameter vector.

According to an embodiment, the Attribute Calculator (AC) 209 extracts time-synchronized sequences of feature vectors from the Spoken Language Processor 104, including ASR Spectral features that may include waveforms, frequency spectra, and similar basic, time varying parameters aligned with phonetic segments identified in the speech signal. The AC 209 compares the incoming features with respect to distributions of values and delta values for those segments and other segment sets established by operation of the FAMO 107 during training. The spectral-modeling module 302 produces a new set of spectral log likelihood parameters, which form part of the time-sequence matrix of values that form the input to the optimized matrix-vector map 320.

According to an embodiment, the Attribute Calculator (AC) 209 extracts a time-synchronized sequence of nominal events from the Spoken Language Processor 104, including ASR Content features that may include linguistic units and other recognized vocal events and similar basic, time dependent events in the speech signal. The AC 209 compares the incoming features with keyword lists, coherence models, and other metrics of nominal quality, some of which based on methods using bag-of-words and/or single value decomposition, as established by operation of the FAMO 107 during training. The content-modeling module 303 produces a new set of content parameters, which form part of the time-sequence matrix of values that form the input to the optimized matrix-vector map 320.

According to an embodiment, the Attribute Calculator (AC) 209 extracts time synchronized sequences of feature vectors from the Spoken Language Processor 104, including ASR duration features that may include durations of linguistic and phonetic units as identified and aligned by the ASR component of SLP (104) with reference to a user speech signal. The AC 209 transforms the incoming features with respect to distributions of expected duration values and distributions of duration values for the linguistic units and for neighboring units established by operation of the FAMO 107 during training. The duration-modeling module 304 produces a new set of parameters, including speech rate, articulation rate, mean run rate, average pause time, pause times with reference to expected pause times at given linguistic structural locations, which form part of the time-sequence matrix of values that form the input to the optimized matrix-vector map 320.

According to an embodiment, the Attribute Calculator (AC) 209 extracts time-synchronized sequences of feature vectors from the Spoken Language Processor 104, including ASR confidence features that may include confidence scores of recognized linguistic and phonetic units as produced by the ASR component of SLP (104) with reference to a user speech signal. The AC 209 scales the incoming features with respect to distributions of expected confidence values established by operation of the FAMO 107 during training. The confidence module 305 produces a new set of parameters, which form part of the matrix of values that form the input to the optimized matrix-vector map 320. According to an embodiment, the Attribute Calculator (AC) 209 extracts time-synchronized sequences of feature vectors from the Touch Processor 105, including touch signal features that may include touches, holds, touch-releases, or any screen-mediated action or movement that qualifies as a touch event with reference to a single visual object or set of manifold visual objects presented on screen or by language. For each sampled time during a behavior segment, the coordinates and time for all single and multiple detected touch is smoothed and transmitted by the Touch Processor 105 to the motoric-modeling module. For example, a user may copy a visually presented line drawing as a series of touch-move-release gestures that are made inside a conventionally displayed “drawing area” on the screen, where each touch-move-release is a touch gesture. The AC 209 groups the incoming touch features into motoric events that relate to the demands of prompting events that may comprise a task. The motoric-model transforms the touch events into motoric features including onsets, accuracy, latency, interval-consistency, offsets, persistence and duration, and scales them with respect to distributions of values established by operation of the FAMO 107 during training. The motoric-modeling module 306 produces a new set of parameters from these scaled values and their summary statistics, including means, medians, modes, quartiles, extremes, moments, delta-sign segments, peaks, linear and quadratic regression coefficients, percentiles, and similar relevant descriptive values, which form part of the time-sequence matrix of values that form the input to the optimized matrix-vector map 320.

According to an embodiment, the Attribute Calculator (AC) 209 extracts time-synchronized sequences of feature vectors from the Touch Processor 105 similar to those received by the motoric model processor, and further including touch content features that may represent virtual operation of on-screen virtual objects including buttons, keys, targets, screen segments, images or texts that provide symbolic or nominal meaning to a touch event. The AC 209 adds features to the incoming feature streams with respect to the known location at all times of the virtual screen objects and the criteria for evaluating a touch event with respect to the extent and location of virtual screen objects. The incoming content feature values are then transformed and scaled with respect to distributions of values established by operation of the FAMO 107 during training. The content-modeling module 307 produces a new set of parameters, including onsets, accuracies, latencies, intervals, offsets, and nominal values of the buttons, keys, targets, screen segments, images or texts, which form part of the time-sequence matrix of values that form the input to the optimized matrix-vector map 320.

According to an embodiment, the Attribute Calculator (AC) 209 extracts time-synchronized sequences of feature vectors from the Video Processor 106, including face movement features that may include visible and trackable units from Ekmam's set of action units in the FACS coding scheme (Ekman & Rosenberg, 2005), along with winks, blinks, eye movements and the movements of larger structures like the user's head and easily visible landmarks on the surface of the user's face. The AC 209 normalizes the incoming features with respect to distributions of values and delta values in the current behavior segment and the distributions established by operation of the FAMO 107 during training. The signal-modeling module 308 produces a new set of face parameters, which form part of the time-sequence matrix of values that are input to the optimized matrix-vector map 320.

According to an embodiment, the Attribute Calculator (AC) 209 extracts time-synchronized sequences of feature vectors from the Video Processor 106, including bodily movement features that may include signals from a body motion tracker implemented within or in series with the video processor. Bodily movements feature changes in posture, voluntary movements of the arms and legs, conventional gestures such as shrugs or acts of pointing. The AC 209 warps the incoming features with respect to distributions of values and delta values established by operation of the FAMO 107 during training. The signal-modeling module 309 produces a new set of parameters which form part of the time-sequence matrix of values that form the input to the optimized matrix-vector map 320.

Based on the received input, the matrix vector map 320 is utilized to analyze participant user attitude, communication skills, energy levels, basic skills and memory, among others. Additionally, attitude is determined by estimating at least one of user outlook, cooperation level, responsibility level and persistence level. According to a preferred embodiment, outlook is graded on a scale that ranges over positive and negative values; accordingly, cooperation, responsibility, and persistence are graded by the attribute calculator based on predefined criteria. Further, the attribute calculator estimates communication skills by determining participant user speaking, listening, vocabulary, diction and articulation skills, and social awareness, etiquette and politeness. Participant user energy is preferably estimated by determining tempo (fast/slow), vigilance, accuracy and precision. Preferably, participant user basic skills are estimated by determining user literacy and numeracy. Preferably, determining user spatial and sequential awareness and narrative articulation, enables estimation of user memory capabilities.

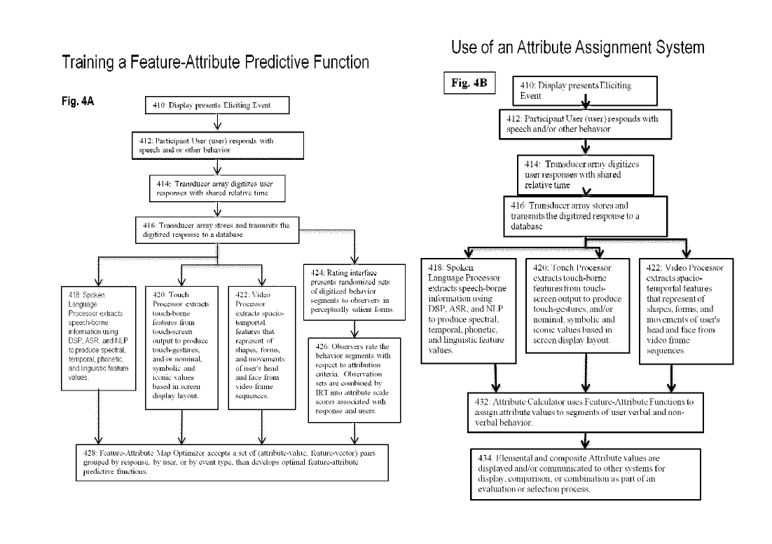

FIG. 4A is an overview of a flow diagram illustrating a step by step process by which Attribute Calculators are determined using both a participant user's digital behavior signals and observer ratings. In Step 410, the system is caused to elicit a response (input) from the participating user. Step 412 allows the participating user to respond via speech or/and other behavioral input. Step 414 includes digitizing user responses with shared relative time. According to an embodiment the digitization is performed via a transducer array. In step 416 the transducer array stores and transmits the digitized response to database 102. Step 418 includes producing spectral, temporal, phonetic, and linguistic feature values, which producing comprises extracting speech-borne information. According to an embodiment, extracting speech-borne information comprises digital signal processing (DSP), ASR, and natural language processing (NLP) to produce the said spectral, temporal, phonetic, and linguistic feature values. Step 420 includes extracting touch-borne features from touch-screen input to produce touch-gestures, and/or nominal, symbolic and iconic values based in screen display layout. Preferred embodiments include a dedicated Touch Processor for extracting touch-borne features. Step 422 includes extracting spatio-temporal features that represent shapes, forms, and movements of user's head and face from video frame sequences. Preferred embodiments comprise a dedicated Video Processor for extracting the spacio-temporal features. Step 424 includes presenting randomized sets of digitized behavior segments to observers in perceptually salient forms, preferably via a rating interface. Step 426 includes, based on observer rated behavior segments with respect to attribution criteria, combining observation sets by Item Response Theory (IRT) into attribute scale scores associated with response, item (event type), and user. Step 428 includes accepting a set of (attribute-value, feature-vector) pairs grouped by response, by user, or by event type, and accordingly developing optimal feature-attribute predictive functions. Preferably the accepting comprises accepting by a dedicated Feature-Attribute Map Optimizer.

FIG. 4B is an overview of a flow diagram illustrating a step by step process by which Session Interactions are automatically rated using both a participant user's digital behavior signals and an Attribute Calculator. The steps 410 to 422, as described above apply to this process, after which step 432 includes assigning attribute values to segments of user verbal and non-verbal behavior. Preferably Attribute Calculator uses Feature-Attribute Functions to assign the attribute values. Step 434 includes displaying and/or communicating elemental and composite Attribute values to other systems for display, comparison, or combination as part of an evaluation or selection process.

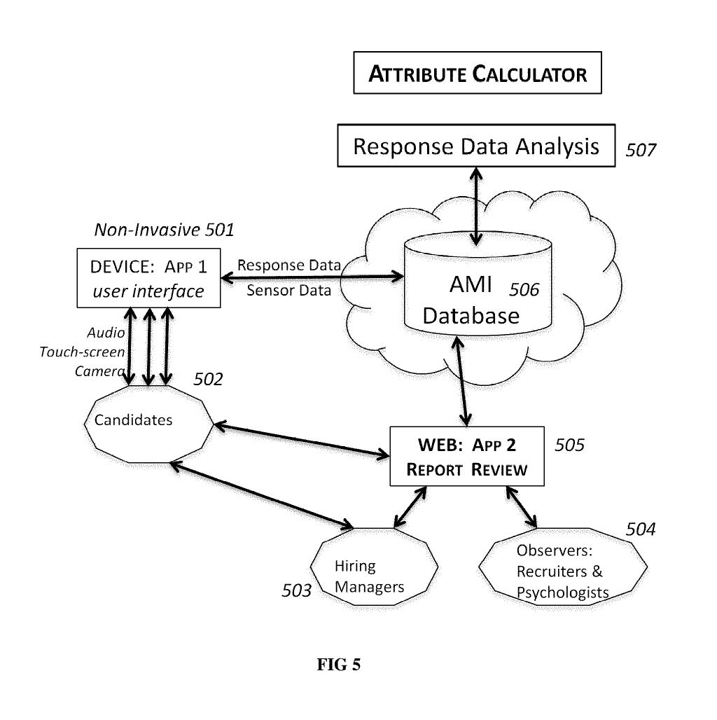

FIG. 5 illustrates a working embodiment of the system and method in a networked environment. Candidates 502 responses to queries and tasks presented via device 501 are recorded through audio, video and touch input capability. Response and sensor data is aggregated in Database 506 and module for response data analysis 507 comprises the core of attribute calculator, analyzes the aggregated response and sensor data. The analyzed response and sensor data, i.e. the calculated attributes are stored in Database 506, and presented via web interface 505 to candidates, hiring managers 503 and observers, i.e. recruiters and psychologists 504.

Embodiments disclosed include computer automated systems and methods that allow quick, short, simple, virtual interviews enabling interviewees to respond to questions and tasks judged most relevant by recruiting professionals and psychologists, and mapping the performance sample to the attributes that are most important to collaborators, co-workers, and hiring managers.

Embodiments disclosed include improved automated estimating systems and methods that accomplish the automated estimating with improved accuracy and reduced time. Embodiments disclosed enable inexpensive, consistent, automated and efficient interview taking and interview results, soliciting and using spontaneous constructed responses as a basis for attribution rating.

Embodiments disclosed include systems and methods configured to evaluate real-time human reaction to unique situations. Embodiments disclosed enable objective measurement of human traits, states and propensities to actions and reactions across a variety of circumstances, including situations that are socially neutral, for which reactions provide emotional valence samples characteristic of participant users.

Embodiments disclosed enable measurement of a person's natural mode of thought through spontaneous speech capture comprising capturing what a person says, how they say it, and how quickly it is said. Embodiments disclosed further enable consistently and accurately detecting and estimating levels of “attitude” and “energy” by capturing at least one of facial expression, content and dynamics of speech, and bodily posture and motion (trunk, head, hands, eyes).

Embodiments disclosed include improved methods and systems via machine learning, sentiment analysis, spoken language processing, effective computing, Human Computer Interfacing (HCI), psychometrics, bio-behavioral analytics, multimodal interaction, emotion detection, consumer research, psychological testing, or computational linguistics, or/and a combination of any or all of the above.

Embodiments disclosed enable inexpensive, accurate, consistent qualitative analysis of “soft skills” such as energy, attitude, and communication. Further, embodiments disclosed enable a non-invasive (respectful) basis for temporary exclusion of people from work or other activity. For example, if a person has an intoxicated or groggy aspect at work, behind the wheel, at a machine, embodiments of the present invention enable and provide justification to exclude the user from physical presence at a site or from an activity. The method is more precise than traditional evaluations, and embodiments of the method are normed to the participant user (person), and not to a general population.

While certain exemplary embodiments have been described and shown in the accompanying drawings, it is to be understood that such embodiments are merely illustrative and not restrictive of the broad invention and that this invention is not limited to the specific constructions and arrangements shown and described, since various other modifications may occur to those ordinarily skilled in the art upon studying this disclosure. In an area of technology such as this, where growth is fast and further advancements are not easily foreseen, the disclosed embodiments may be readily modifiable in arrangement and detail as facilitated by enabling technological advancements without departing from the principals of the present disclosure or the scope of the accompanying claims.

Since various possible embodiments might be made of the above invention, and since various changes might be made in the embodiments above set forth, it is to be understood that all matter herein described or shown in the accompanying drawings is to be interpreted as illustrative and not to be considered in a limiting sense. Thus it will be understood by those skilled in the art that although the preferred and alternate embodiments have been shown and described in accordance with the Patent Statutes, the invention is not limited thereto or thereby.

The figures illustrate the architecture, functionality, and operation of possible implementations of systems and methods according to various embodiments of the present invention. It should also be noted that, in some alternative implementations, the functions noted/illustrated may occur out of the order noted in the figures. For example, two blocks shown in succession may, in fact, be executed substantially concurrently, or the blocks may sometimes be executed in the reverse order, depending upon the functionality involved.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

In general, the steps executed to implement the embodiments of the invention, may be part of an automated or manual embodiment, and programmable to follow a sequence of desirable instructions.

The present invention and some of its advantages have been described in detail for some embodiments. It should be understood that although some example embodiments of the system and method are described with reference to user state estimating and assessing for interview purposes, the system and method disclosed is reconfigurable, and embodiments include systems that may be dynamically adapted to be used in other contexts as well. It should also be understood that various changes, substitutions and alterations can be made herein without departing from the spirit and scope of the invention as defined by the appended claims. An embodiment of the invention may achieve multiple objectives, but not every embodiment falling within the scope of the attached claims will achieve every objective. Moreover, the scope of the present application is not intended to be limited to the particular embodiments of the process, machine, manufacture, and composition of matter, means, methods and steps described in the specification. A person having ordinary skill in the art will readily appreciate from the disclosure of the present invention that processes, machines, manufacture, compositions of matter, means, methods, or steps, presently existing or later to be developed are equivalent to, and fall within the scope of, what is claimed. Accordingly, the appended claims are intended to include within their scope such processes, machines, manufacture, compositions of matter, means, methods, or steps.

We claim:

1. A computer automated system, comprising a processing unit, a non-transitory storage element coupled to the processing unit and having instructions encoded thereon, which instructions when implemented by the processing unit cause the computer automated system to:
capture a user input comprising at least one of a speech input and a touch input;
combine the captured speech and touch input in at least one of linear and non-linear form, and based on the combined speech and touch input, generate a state estimate;
wherein the generated state estimate comprises extracting a single or plurality of user attributes; and
based on the extracted single or plurality of user attributes, assess dimensions of the user interpersonal proficiency.

2. The computer automated system of claim 1 wherein the generated state estimate is based on predefined criteria comprising an aggregated plurality of state estimates.

3. The computer automated system of claim 1 wherein the computer automated system is further caused to:
in capturing the user input, capture large muscle movements via a single or plurality of accelerometers, a muscle contraction, an eye movement and a gaze; and
in generating the state estimate, combine the captured large muscle movements with the eye movement, and the speech and touch responses.

4. The computer automated system of claim 1 wherein the computer automated system is further caused to, in capturing the user speech and touch input:
capture speech and touch response latencies;
capture the user rate of responding by speech or touch in a single or plurality of interactive tasks; and
capture the accuracy and time-course of active touch sequences used in the touch.

5. The computer automated system of claim 1 wherein assessing the user psychological state further comprises determining the user attitude and energy level, which comprises combining captured speech with the user motion captured on a touch screen according to pre-defined reference criteria.

6. The computer automated system of claim 1 wherein the generated state estimate is based on at least one of the user response time combined in linear or non-linear form, a single or plurality of automatically assigned tasks, and a single or plurality of response parameters.

7. The computer automated system of claim 1 wherein the generated state estimate comprises a user attribute.

8. A computer implemented method comprising:
capturing a user input comprising at least one of a speech input and a touch input;
combining the captured speech and touch input in at least one of linear and non-linear form, and based on the combined speech and touch input, generating a state estimate;
wherein the generated state estimate comprises extracting a single or plurality of user attributes; and
based on the extracted single or plurality of user attributes, assessing the user psychological state.

9. The computer implemented method of claim 8 wherein the generated state estimate is based on predefined criteria comprising an aggregated plurality of state estimates.

10. The computer implemented method of claim 8 further comprising:
in capturing the user input, capturing large muscle movements via a single or plurality of accelerometers, a muscle contraction, an eye movement and a gaze; and
in generating the state estimate, combining the captured large muscle movements with the eye movement, and the speech and touch responses.

11. The computer implemented method of claim 8 further comprising:
in capturing the user speech and touch input, capturing the user speech and touch input response latencies;
capturing the user rate of responding by touch or drawing in a single or plurality of interactive tasks; and
capturing the accuracy and time-course of active touch sequences used in drawing.

12. The computer implemented method of claim 8 wherein assessing the user psychological state further comprises determining the user attitude and energy level, which comprises combining captured speech with the user motion captured on a touch screen according to a pre-defined reference criteria.

13. The computer implemented method of claim 8 wherein the generated state estimate is based on at least one of the user response time combined in linear or non-linear form, a single or plurality of automatically assigned tasks, and a single or plurality of response parameters.

14. The computer implemented method of claim 8 wherein the generated state estimate comprises a user attribute.
